(19) United States
(12) Patent Application Publication
(10) Pub. No.: US 2007/0238118 A1
(43) Pub. Date: Oct. 11, 2007

(54) METHODS AND KITS FOR SEQUENTIALLY ISOLATING RNA AND GENOMIC DNA FROM CELLS

(75) Inventor: Marianna M. Goldrick, Austin, TX (US)

Correspondence Address: AMBION, 2130 WOODWARD STREET, AUSTIN, TX, 78744-1832, US

(73) Assignee: Ambion, Inc., Austin, TX (US)

(21) Appl. No.: 11/697,686

(22) Filed: Apr. 6, 2007

Related U.S. Application Data
(60) Provisional application No. 60/790,374, filed on Apr. 6, 2006.

(51) Int. Cl. C12Q 1/68; C07H 21/04

(52) U.S. Cl. 435/6; 536/25.4

(57) ABSTRACT

Methods for obtaining nucleic acid from nucleated cells are disclosed, wherein RNA and fragmented genomic DNA can be sequentially obtained from the same starting material are disclosed. In some embodiments, protein can also be obtained from the same starting material as the RNA and DNA are obtained. According to certain methods, whole blood or a blood fraction comprising nucleated cells is combined with a capture surface and at least some of the leukocytes are retained on the surface. The RNA is released from the retained leukocytes, then the capture surface is treated with a suitable nuclease or other DNA fragmenting agent to release DNA fragments. In certain embodiments, either the released RNA, the released DNA, or both the released RNA and the released DNA are employed in one or more molecular biology application.

### CROSS-REFERENCE TO RELATED APPLICATIONS

This application claims priority under 35 U.S.C. §119 to U.S. Provisional Application Ser. No. 60/790,374, filed Apr. 6, 2006, the disclosure of which is incorporated herein by reference for any purpose.

### FIELD

The present teachings generally relate to methods, reagents, and kits for obtaining RNA and DNA sequentially from vertebrate cells, typically mammalian cells.

### INTRODUCTION

Many current molecular biology techniques comprise evaluating the DNA, the RNA, and/or the proteins obtained from a sample to evaluate the state of the individual organism from which the sample was obtained. For example gene expression profiling and miRNA expression profiling are commonly used methods to evaluate RNA. Various DNA-based molecular biology techniques for example but not limited to, genotyping applications, methylation analysis, and other epigenetic analyses are frequently employed. In many circumstances it would be desirable to obtain DNA and RNA from the same sample in order to correlate results. Currently, the opportunity to obtain DNA and RNA from the same sample is limited.

Blood is an attractive tissue for obtaining biological material such as DNA, RNA and proteins because it is an actively metabolizing human tissue that is routinely accessible and expendable. Also, since circulating blood leukocytes come into intimate contact with all internal organs, they may be able to act as sentinel cells to monitor systemic pathological conditions. Many recent reports describe mRNA expression patterns determined in whole blood or in fractionated blood leukocytes that correlate with systemic conditions including neurological disease, cancer, asthma, autoimmune disease, etc. A simple method for isolating both RNA and DNA from the same sample would be useful for providing starting nucleic acid for a variety of molecular biology techniques.

### SUMMARY

The present teachings are directed to methods, reagents, and kits for isolating nucleic acid from cells. Methods and kits for obtaining nucleic acid from nucleated cells, wherein first RNA and then fragmented genomic DNA can be obtained one after the other from the same starting material are disclosed. In some embodiments, proteins are also obtained from the same starting material as the isolated RNA and DNA. According to certain disclosed methods, whole blood is combined with a capture surface, for example but not limited to, a leukocyte depletion filter (LDF) and nucleated cells such as leukocytes are retained on or in the capture surface while red blood cells, serum proteins, and other blood components are not typically retained. The retained cells are lysed using chemical and/or physical techniques known in the art, releasing the cellular RNA which is removed and typically isolated. One or more nuclease is then combined with the RNA-depleted capture surface and DNA is released. In certain embodiments, serum proteins may also be obtained from the same blood sample as the RNA and the DNA.

According to certain methods, whole blood or a blood fraction comprising nucleated cells is combined with a capture surface and at least some of the nucleated cells, typically leukocytes, are retained on the surface. Typically neither the serum proteins nor the red blood cells are retained on the surface. The RNA is released from the retained leukocytes, then the capture surface is treated with a suitable nuclease or other DNA fragmenting agent to release DNA fragments. In certain embodiments, either the released RNA, the released DNA, or both the released RNA and the released DNA are employed in one or more molecular biology application.

The DNA obtained is typically fragmented; some of the DNA recovered has a size distribution of several hundred bp but most of the obtained DNA fragments are larger, ranging up to greater than 2 kilobases in size. The DNA fragments are especially useful for PCR amplification based techniques, which is expected to allow detection of point mutations, SNPs, and other types of mutations (e.g., translocations, insertions/deletions) that are found in circulating white blood cells (WBCs), sometimes referred to as leukocytes. The capture surface may also trap circulating epithelial cells, which could arise from metastatic disease (see, e.g, Yamada et al., Int. J. Clin. Oncol. 2:143-46, 1997). Thus the RNA and/or DNA isolated according to the current teachings may be useful for detection of genetic changes associated with cancer. A particular advantage of certain disclosed methods is the sequential isolation of total RNA, including without limitation small non-coding (ncRNA, for example but not limited to, microRNA (miRNA) and small interfering RNA (siRNA)), and genomic DNA from the same blood sample.

In certain embodiments, the mixture comprising fragmented DNA and at least one nuclease is collected into or subsequently combined with an inactivating agent. Exemplary inactivating agents include guanidinium thiocyanate, guanidine hydrochloride, sodium thiocyanite, urea, or other chaotropes that are capable of denaturing or otherwise reducing the enzymatic activity of at least one nuclease in the mixture; a protease, for example but not limited to, proteinase K; an antibody molecule or fragment thereof, or an aptamer that can bind to the releasing enzyme and at least partially block its activity on the DNA fragments. Those in the art will appreciate that any compound that is capable of reducing the degradation of the DNA in the mixture may be a suitable inactivating agent. Typically such compounds are capable of decreasing the activity of at least one nuclease in the mixture. In some embodiments, the inactivating agent is in solid form while in other embodiments of the instant methods, the inactivating agent is in a liquid form. According to certain methods, fragmented DNA is released from an RNA-depleted capture surface using a nuclease solution and the mixture comprising the nuclease solution and the fragmented DNA are collected in one or more container comprising at least one inactivating agent. In certain embodiments, one or more nuclease in the mixture comprising the fragmented DNA is physically inactivated such as by heating the mixture, wherein such physical inactivation includes at least decreasing the degradation of the fragmented DNA relative to the same method without the physical inactivation step.

According to certain disclosed methods, the obtained DNA is typically fragmented; most (˜80%) of the DNA recovered has a size distribution ranging between 1.5 kb and 3 kb, and the remainder is larger and smaller than this range. The size distribution of the DNA fragments can be modulated by manipulating various reaction conditions, for example but not limited to, the properties of the specific nuclease(s) employed, the concentration and volume of nuclease solution, and by the length of time that the RNA-depleted capture surface comprising DNA is exposed to the nuclease solution. The DNA fragments are especially useful as input material for amplification using PCR, which may be used for a variety of downstream applications, including without limitation, detection of point mutations, single nucleotide polymorphisms (SNPs), and other types of mutations (e.g., translocations, insertions/deletions, duplication of large chromosomal regions) that can be found in circulating WBCs. The DNA obtained according to the disclosed methods is also useful for monitoring epigenetic events such as methylation, which may be dysregulated in pathological conditions. Likewise, the RNA obtained according to certain of the instant methods can also be used for a number of downstream molecular biology applications, for example but not limited to, gene expression profiling and miRNA expression profiling. In some embodiments, the capture surface may also retain circulating epithelial cells, which could arise from metastatic disease, thus the obtained DNA and RNA would be useful for detection of genetic changes associated with cancer.

A particular advantage of certain disclosed methods is that they allow for the sequential extraction of total RNA and genomic DNA from cell population, including without limitation cells from the same blood sample. When the capture surface comprises a leukocyte depletion media, for example but not limited to, the filter of the LeukoLOCK™ Kit (Ambion), blood filtrate that passes through is substantially depleted of leukocytes. The red blood cells in the filtrate can readily be separated from the plasma fraction using a number of generally known techniques, such as centrifugation. The resulting purified plasma component can be used for standard blood chemistry tests typically carried out on blood plasma, including assessment of non-protein analytes such as glucose and uric acid, and detection of protein analytes such as cardiac enzymes, liver enzymes, prostate specific antigen (PSA), and the like.

Kits for performing certain of the instant methods are also disclosed.

These and other features of the present teachings are set forth herein.

### DRAWINGS

The skilled artisan will understand that the drawings, described below, are for illustration purposes only. These figures are not intended to limit the scope of the present teachings in any way.

FIG. 1: Schematically an exemplary assembly employed in certain embodiments of the current teachings. In this exemplary assembly, “Filter” refers to a LeukoLOCK™ filter assembly (see, e.g., LeukoLOCK™ Total RNA Isolation System (Ambion Cat.# 1923).

FIG. 2: depicts the results of an analysis of DNA fragments obtained according to one embodiment of a method of the current teachings, wherein the DNA fragments are obtained using a nuclease digestion solution comprising DNase I, as described below. The obtained fragments are analyzed using an Agilent 2100 Bioanalyzer instrument using a standard protocol. The x-axis indicates fragment size in nucleotides (shown as [nt]) and the y-axis indicates fluorescent intensity of the labeled fragments measured in fluorescence units (shown as [FU]). As shown by the arrow, the size of the fragments at the peak of the Bioanalyzer trace are 1827 nucleotides.

FIG. 3: depicts the results of the analysis of DNA fragments obtained according to another method of the current teachings, wherein the DNA fragments are obtained using a nuclease digestion solution comprising benzonase, as described below. The obtained fragments are analyzed using an Agilent 2100 Bioanalyzer instrument. The concentration of the isolated DNA was 175.4 ng/μL. The arrows indicate the approximate location of DNA fragments that are approximately 1000 and 2500 nucleotides long, respectively.

FIG. 4: depicts the results of the analysis of DNA fragments obtained according to another method of the current teachings using an Agilent 2100 Bioanalyzer instrument. The DNA fragments in this example were obtained using a nuclease inactivating agent comprising 0.5 M EDTA at pH 8, as described below.

FIG. 5: depicts the results of the analysis of DNA fragments obtained according to another embodiment of disclosed methods using an Agilent 2100 Bioanalyzer instrument. The DNA fragments in this example were obtained using a lysis solution comprising 8 M urea and 250 mM NaCl, as described below.

FIG. 6: depicts the results of the analysis of DNA fragments obtained according to another embodiment of disclosed methods using an Agilent 2100 Bioanalyzer instrument. The DNA fragments in this example were obtained using a lysis solution comprising 2% sodium dodecyl sulfate, as described below.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including patents, patent applications, articles, books, treatises, and internet web pages are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The term “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

Lysing refers to any process in which the integrity of the cellular membrane of a cell is compromised to the point that at least some of the cellular contents, including but not limited to RNA, are released. Those in the art will understand that any number of well known chemical or physical cell lysis techniques can be used in the current teachings. In some embodiments, a lysis solution comprising one or more detergent is employed to lyse the retained cells, including without limitation, solutions comprising nonionic detergents, anionic detergents, cationic detergents, zwitterionic detergents, or combinations thereof. In certain embodiments, solutions comprising chaotropes are employed to lyse cells, for example but not limited to solutions comprising guanidinium thiocyanate, guanidine hydrochloride, potassium thiocyanate, urea, or the like, and including combinations thereof. In certain embodiments, a solution comprising at least one detergent and at least one chaotrope is used to lyse the retained cells. In some embodiments, cells are lysed by physical forces such as shear forces, osmotic shock, or sonication using methods and apparatuses known in the art (see, e.g., Belgrader et al., Clin. Chem. 47(10):1929-31, 2001).

The term “nuclease” as used herein, refers to any polypeptide that when combined with a capture surface comprising retained nucleic acid is capable of freeing at least part of one or more such retained nucleic acids through a catalytic process. Exemplary nucleases include endonucleases and exonucleases for example but not limited to, DNases from a wide variety of eukaryotic or prokaryotic species or viruses and restriction endonucleases, typically from eubacterial and archaeal species. Those in the art will appreciate that the fragment size of the DNA obtained according to the instant methods is dependent on a number of factors such as the nuclease employed and its concentration, exposure time, reaction conditions for example but not limited to the pH and buffering capacity of the digestion solution, the presence and concentration of certain cations such as Mg²⁺, and temperature. Those in the art will understand that DNA fragment size can be increased or decreased by manipulating these factors during routine experimentation.

### III. Exemplary Embodiments

According to certain disclosed methods, nucleated cells such as leukocytes (also known as white blood cells or WBCs) and in some instances circulating epithelial cells are separated from red blood cells and other blood components, for example but not limited to, serum proteins, using a capture surface. RNA and genomic DNA (gDNA) fragments are isolated from the residual material retained on the capture surface, such as a leukocyte depletion filter used in the LeukoLOCK™ kit (Ambion, Austin, Tex.). In some embodiments, the retained cells are combined with an RNA stabilizing agent, for example but not limited to, RNAlater® Solution (U.S. Pat. Nos. 6,528,641 and 6,204,375) then the retained cells are lysed using a lysis solution, releasing the RNA but typically substantial amounts of DNA are not released. In other embodiments, particularly when RNA recovery is not important, the RNA is released from the retained cells without treatment with a nucleic acid stabilizing agent. Following release of the RNA, the capture surface is exposed to one or more nuclease, a nucleic acid fragmenting agent, for example but not limited to, sodium hydroxide, or combinations thereof, and DNA fragments are released from the RNA-depleted capture surface and recovered.

Those in the art will appreciate that using whole blood as a starting material, the disclosed methods and kits allow RNA, including miRNA and other small ncRNA to be isolated. According to certain methods of the current teachings, fragmented gDNA is subsequently obtained from the same whole blood sample by treating the RNA-depleted nucleic acid with certain endonucleases under conditions suitable to fragment the remaining gDNA, inactivating the endonucleases, and isolating the gDNA fragments.

In some embodiments, the capture surface comprises a porous membrane or filter, comprising a suitable material for separating WBCs from red blood cells (RBCs), such as a silica-based material including but not limited to glass fiber filter or a glass frit or a separation media comprising one or more organic polymer. In some embodiments, the capture surface is non-porous, such as certain planar surfaces or non-planar surfaces, including without limitation, beads or particles, for example but not limited to, glass beads, coated magnetic or paramagnetic beads, and beads comprising polymers such as agarose, polyacrylamide, and other polymers. In certain embodiments, the capture surface comprises one or more polymers and is granular, fibrous, porous, or combinations thereof. Those in the art will appreciate that the shape and composition of the capture surface is not a limitation of the current teachings provided that, under appropriate conditions, at least some cells from a starting material are retained on or in the material comprising the capture surface, the retained cells can be lysed and the RNA and then the DNA can be obtained.

In some embodiments, the capture surface comprises a leukocyte depletion filter. Such filters and leukocyte depletion media are well known to those in transfusion medicine to remove most or substantially all of the leukocytes from whole blood prior to certain medical procedures. Leukocyte depletion filters and leukocyte depletion media are commercially available from a number of sources and typically comprise a variety of silica-based and/or polymeric materials, for example but not limited to, glass fibers, polyester fibers, nonwoven or microfiber glass coated with polysaccharide. Polyethylene terephtalate (PET), polyamide, polyester, polypropylene, polyvinyl alcohol, polyvinylidene difluoride, polytetrafluoroethylene, or combinations thereof may be used (see, e.g., U.S. Pat. Nos. 6,645,388; 6,670,128; 6,337,026; and 5,451,321; Henschler et al., Ann. Hematol. 84:538-44, 2005; and deVries et al., Ann. Cardiac Anesth. 8:117-24, 2005). Leukocyte depletion filters and media are commercially available from a variety of sources, for example, Ambion, Inc., HemaSure, Inc., Pall Corporation, and Baxter Healthcare Corporation.

One useful source of capture surfaces, particularly when the desired cells are present in whole blood, are the leukocyte depletion matrices sold by Pall Corporation, under the name Leukosorb™ Medium. Leukosorb is a fibrous medium that was originally designed for the depletion of WBCs from blood for transfusion. Descriptions of the Leukosorb products and their use may be found in U.S. Pat. Nos. 5,501,795, 5,100,564, 4,880,548, 4,923,620, 4,925,572, 5,229,012, and 5,344,561, as well as U.S. Patent Application No. 2003/0134417.

In certain embodiments of the instant methods, after the leukocytes are bound, trapped, or otherwise retained on or in the capture surface, the surface comprising the leukocytes is washed to remove residual RBCs and other blood components. In certain embodiments, the blood fraction that passes through the capture surface is further fractionated to separate the RBCs from blood plasma. In some embodiments, the serum proteins or other analytes contained in the blood plasma are further analyzed and in yet other embodiments, information obtained from the analysis of the blood plasma is compared with data obtained from the nucleic acid fractions obtained from the leukocytes retained on the capture surface.

According to certain disclosed methods, after the RNA has been eluted from the support, the bound DNA is fragmented and the fragments are isolated. In certain embodiments, the RNA-depleted capture surface is exposed to a suitable endonuclease, including but not limited to DNase I, under conditions suitable to fragment and release the gDNA that is bound to or trapped within the capture surface. In certain embodiments, where the RNA-depleted capture surface comprises a leukocyte depletion filter, the nuclease digestion solution is slowly passed through the filter to allow the bound gDNA to become fragmented. The flow-through comprising the gDNA fragments is collected, and in certain embodiments combined with a nuclease inactivation agent, including without limitation, EDTA at a suitable concentration, for example but not limited to, 500 mM EDTA, and heated to inactivate the endonuclease. The gDNA fragments are then isolated using any suitable method, for example sodium acetate-alcohol precipitation.

Other endonucleases may be used in the instant methods and kits, including without limitation, benzonase (a non-specific bacterial nuclease), S1 nuclease (a fungal nuclease), micrococcal nuclease (from gram-positive bacteria), mung bean nuclease (a plant nuclease), and restriction endonucleases (including but not limited to nucleases from eubacteria and archea), for example Hpa II, Mbo I, and Mse I, that have four base pair (bp) recognition sites, and combinations of restriction endonucleases having different recognition sites. Different types, combinations, and concentrations of nucleases may be used to modulate the yield and size distribution of DNA isolated from the RNA-depleted capture surface. Different nuclease reaction conditions including variations in pH, divalent and monovalent cations, and temperature may be used to modulate the yield and size distribution of DNA recovered from the capture surface and are also within the scope of the current teachings.

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

In certain embodiments, kits comprise at least one nuclease, for example but not limited to, DNase I, benzonase, S1 nuclease, micrococcal nuclease, mung bean nuclease, a bacterial restriction endonuclease, an archaeal restriction endonuclease, or combinations thereof; at least one capture surface; at least one chemical lysing agent, for example but not limited to, a solution comprising a detergent, a chaotrope, or a detergent and a chaotrope. In certain embodiments, kits comprise at least one anionic detergent, at least one cationic detergent, at least one nonionic detergent, at least one zwitterionic detergent, or combinations thereof; for example but not limited to, sodium dodecyl sulfate (SDS), Triton X-100 (also known as octyl phenol ethoxylate and octylphenolpoly(ethyleneglycolether)ₓ), sarkosyl (also known as N-lauroylsarcosine), and CHAPS. Descriptions of suitable detergents for use in the current methods and kits can be found in, among other places, Biological Detergents, Guide for solubilization of membrane proteins and selecting tools for detergent removal, EMD Biosciences, CB0733-2006USD. Certain kit embodiments comprise an inactivating agent, a nucleic acid stabilizing agent, or an inactivating agent and a stabilizing agent. In certain embodiments, the nucleic acid stabilizing agent is RNAlater® Solution (Ambion, Austin Tex.). In certain embodiments, kits include a lysing solution comprising an organic solution such as Trizol or TRI Reagent® (Ambion); a chaotrope, for example but not limited to, guanidium, guanidine, thiocyanate, and urea; or combinations thereof.

The current teachings, having been described above, may be better understood by reference to examples. The following examples are intended for illustration purposes only, and should not be construed as limiting the scope of the teachings herein in any way.

### EXAMPLES

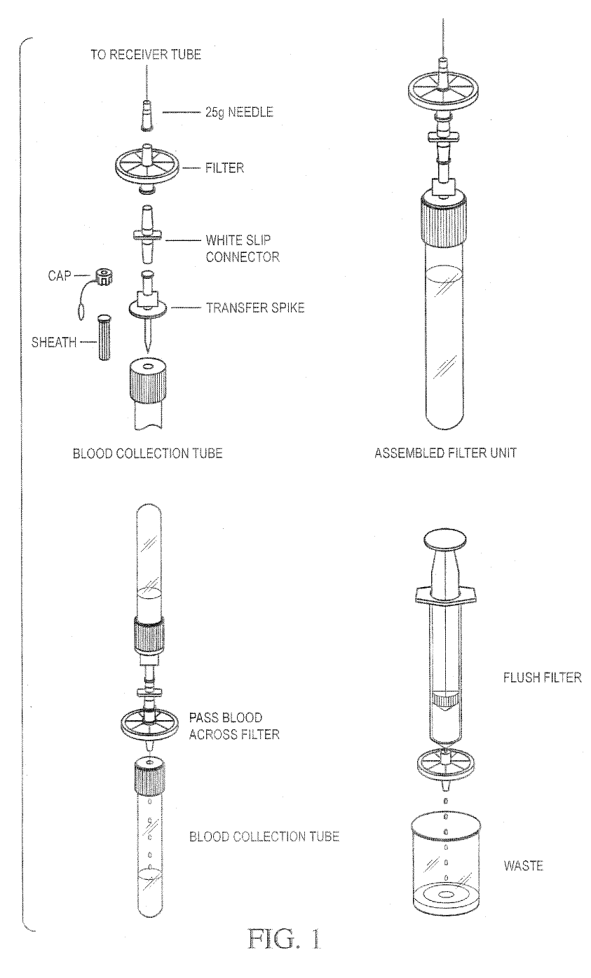

According to some disclosed methods, anticoagulated whole blood was initially processed to capture the WBCs on a leukocyte depletion filter (LDF; an exemplary capture surface) and a nucleic acid stabilizing agent was passed through the LDF to stabilize the RNA in the retained cells. Blood was drawn from a human donor into evacuated blood collection tubes, according to standard phlebotomy procedures. Typically the blood collection tubes contain an anticoagulant, for example but not limited to, potassium EDTA, sodium EDTA, heparin, or sodium citrate. At least a portion of the anti-coagulated whole blood was passed through the LDF to eliminate the red blood cells and blood plasma, and retain the nucleated cells in the blood sample, that contain RNA and DNA, including but not limited to leukocytes and in some instances, circulating epithelial cells. An exemplary assembly for expediting certain steps of some disclosed methods is depicted schematically in FIG. 1 for reference. Referring to the panel on the upper right of FIG. 1, the LDF was contained in a plastic housing designed to connect to standard syringes via a luer fitting at the inlet port, and the tube of blood was connected to the luer fitting via a plastic adaptor (“transfer spike”) and a second plastic connector (“slip connecter”). The pointed side of the transfer spike was inserted through the rubber septum in the cap of the blood tube, and the other side of the transfer spike connected via the slip connector to the inlet port of the LDF device. The outlet port of the LDF device was connected to the hub of a standard 25 g needle, see the upper left panel FIG. 1. As depicted in the panel on the lower left of FIG. 1, the 25 g needle was used to pierce a second empty 10 ml evacuated blood collection tube, which provided the vacuum used to draw the blood sample through the LDF. The leukocytes and certain other nucleated cells are retained on the capture surface and the red blood cells and other blood components pass through the filter. In certain embodiments, the plasma portion of the first filtrate is separated from the red blood cells, typically by centrifugation, and the plasma portion is subjected to downstream analysis. Other formats for retaining cells on a capture surface have also been employed as part of some of the instant methods, for example, an LDF has been attached directly to a 10 ml syringe containing the anticoagulated blood and the plunger of the syringe depressed to provide sufficient pressure to allow at least some of the nucleated cells to be retained on the capture surface and for the red cells and other components to pass through the LDF. As depicted in the lower right panel of FIG. 1, the nucleic acid in the cells retained on the capture surface was stabilized by flushing RNAlater® Solution (Ambion cat#7020) through the capture surface using a standard syringe attached to the inlet port of the LDF. Once the nucleic acid has been stabilized, it has been possible to store the LDF comprising the retained cells for several days to weeks, if desired. When using the exemplary assembly shown in FIG. 1, prior to storage of the filter assembly comprising the stabilized retained cells, the inlet and outlet ports were sealed with small plastic plugs so that the capture surface comprising the retained cells remained exposed to the RNAlater® Solution during storage.

To recover RNA from the retained cells, the capture surface comprising the retained cells is combined with an appropriate lysis solution. In some embodiments this is performed by flushing the LDF comprising the retained cells with an appropriate lysis solution. Exemplary lysis solutions include but are not limited to TriReagent® (a solution comprising guanidinium-thiocyanate and phenol; Ambion; U.S. Pat. Nos. 5,346,994 and 5,945,515) and Lysis Solution (Ambion cat #8540G). Combining the capture surface with an appropriate lysis solution disrupts the membranes of the retained cells, releasing the RNA from at least some of the retained cells into the lysis solution, while the DNA is retained on the capture surface. The released RNA may be further purified for downstream applications using techniques well known in the art, for example using the reagents and protocol in the Ambion LeukoLOCK™ kit (cat # 1923). According to the instant methods, the cellular DNA may be subsequently recovered as a separate fraction, essentially free from RNA.

In certain embodiments, to obtain DNA from the RNA-depleted capture surface, the LDF which was previously combined with Ambion Lysis Solution or with TRI Reagent® (also known as Trizol) as described above, was combined with nuclease digestion solution, for example but not limited to, 2 ml of a solution containing bovine pancreatic DNase 1 (Ambion cat # 2222) in DNase 1 buffer (Ambion cat # 8170G). This nuclease digestion solution further comprises divalent cations such as Mg²⁺ and Ca²⁺, that enhance the activity of DNase I. Those in the art will appreciate that various types and concentrations of nuclease(s) may be used to fragment and obtain the DNA, but for this particular DNase 1, a range of 50-500 units/ml is typically employed. The volume of DNase digestion solution can vary but is typically in the range of 1-5 ml per capture surface.

In one example, the nuclease digestion solution comprised 50 μL of DNase I (2 U/μL, Ambion cat # 2222), 200 μL 10× DNase buffer and 1.75 mL nuclease-free water (Ambion cat #9938). This nuclease digestion solution was combined with the RNA-depleted LDF by attaching it to a 3 ml syringe loaded with the DNase digestion solution. The plastic device containing the LDF included a standard luer fitting allowing for its secure attachment to standard plastic syringes. The plunger of the syringe was depressed to force the solution through the device. The DNase digestion solution was combined with the capture surface using gentle pressure and at a rate of several drops per second, so that it took approximately 10 seconds for the entire volume to pass through the LDF. The filtrate was collected in a 15 mL plastic centrifuge tube that contained 1 gm of solid guanidinium thiocyanate (GuSCN) as an inactivating agent. The tube was closed and inverted several times to dissolve the solid GuSCN and inactivate the nuclease. Then the tube was opened and the contents mixed with one-half volume (1.1 mL) of 100% ethanol. The DNA was recovered from this mixture by solid phase extraction onto a silica filter using the components and reagents from the RNAqueous® kit (Ambion cat #AM 1912). The mixture comprising the inactivated nuclease and the obtained DNA was combined with the silica filter by loading successive 700 μL aliquots into the plastic device containing the filter (i.e. the filter cartridge), which was inserted into a 2 mL collection tube. The assembly was centrifuged for about 10 seconds at about 8,000 rpm in a microcentrifuge and the filtrate decanted from the collection tube into a waste container. The silica filter was then washed once with 0.7 mL of Wash Soln 1 (70% ethanol and 30% Lysis Solution from the RNAqueous® Kit) and twice with 0.7 mL each of Wash Soln 2 (80% ethanol and 50 mM sodium chloride), where each wash was loaded into the filter cartridge and passed through the filter by brief centrifugation. After the filtrate from the last wash was decanted, the filter cartridge was replaced in the collection tube and centrifuged for 1 min at 13,200 rpm to remove the residual fluid. The filter cartridge was transferred to a fresh collection tube for elution of the DNA. The DNA was eluted by adding 0.2 ml of nuclease-free water containing 0.1 mM EDTA that was preheated to 80° C. to the center of the silica filter, storing the assembly for 1 min at room temp, and then centrifuging the assembly for 1 min at 13,200 rpm.

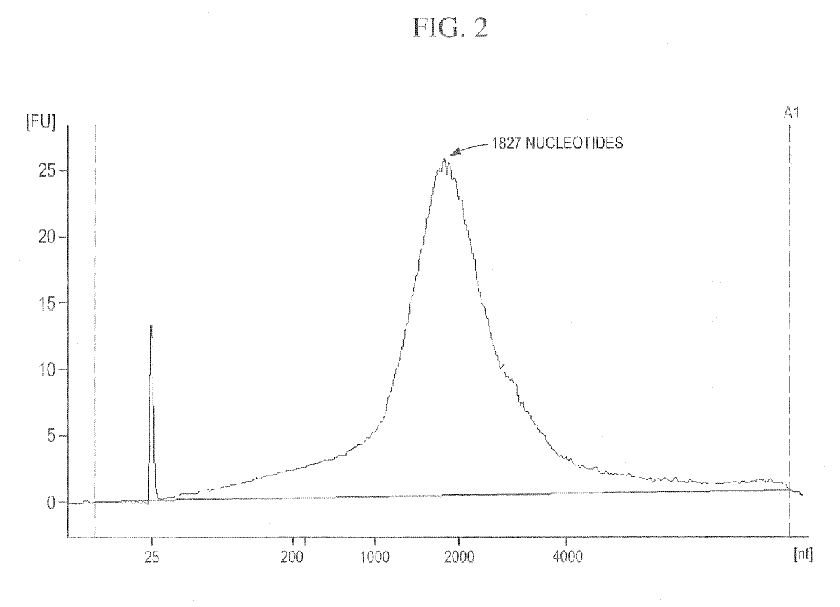

The isolated DNA was analyzed on an Agilent 2100 Bioanalyzer to evaluate the size of the DNA fragments that were obtained. As shown in FIG. 2, the majority of the obtained DNA fragments ranged in size between about 1 kilobase to about 4 kilobase (approximately 80% of the area under the curve is between 1000 and 4000 nucleotides).

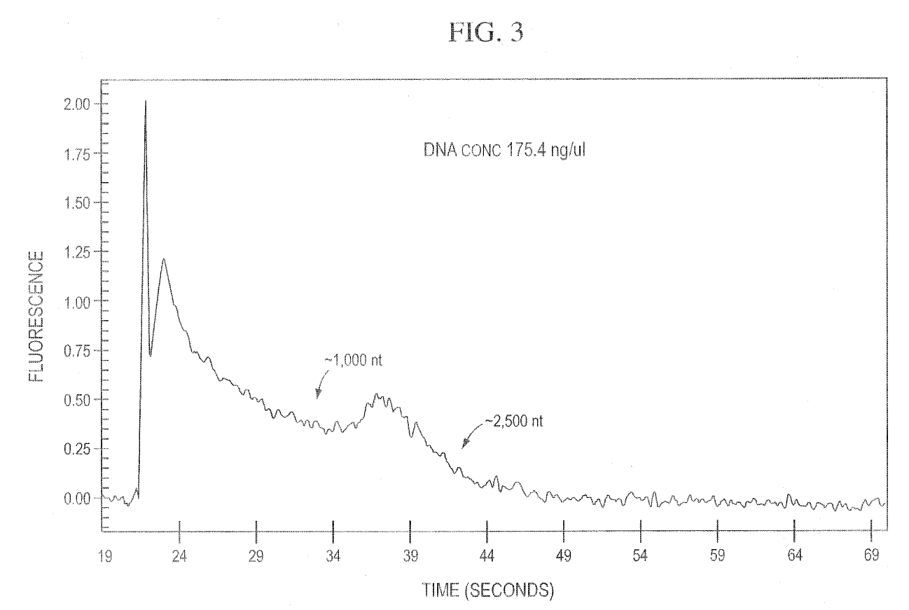

Alternatively, a nuclease digestion solution comprising benzonase, a non-specific bacterial nuclease was employed to fragment DNA retained on the capture surface. Ten mL of anti-coagulated blood was filtered over a LDF capture surface and certain nucleated cells including leukocytes were retained, as described above. The capture surface was combined with RNAlater® Solution to stabilize the RNA in the retained cells and then with TRI Reagent® to lyse the retained cells and release the RNA, as described above. The RNA-depleted capture surface was then combined with a 2 mL a solution comprising 0.5 units/μL benzonase (Merck, Cat. 1.01653.0002) prepared in 1× DNase I buffer. The benzonase filtrate containing the released DNA was collected and processed as described in the previous example to recover the DNA, except that the benzonase filtrate was passed back over the LDF a second time before mixing it with the solid GuSCN, and the DNA was eluted from the silica filter with 100 μL of Elution Solution instead of 200 μL, as described above. The DNA was analyzed using an Agilent 2100 Bioanalyzer. The concentration of the isolated DNA was 175.4 ng/μL. The Bioanalyzer results are shown in FIG. 3. The approximate location of the DNA fragments corresponding to 1000 nucleotides and to 2500 nucleotides are marked by arrows, indicating that a large percentage of the DNA fragments obtained using this method were between 500 and 3000 nucleotides long.

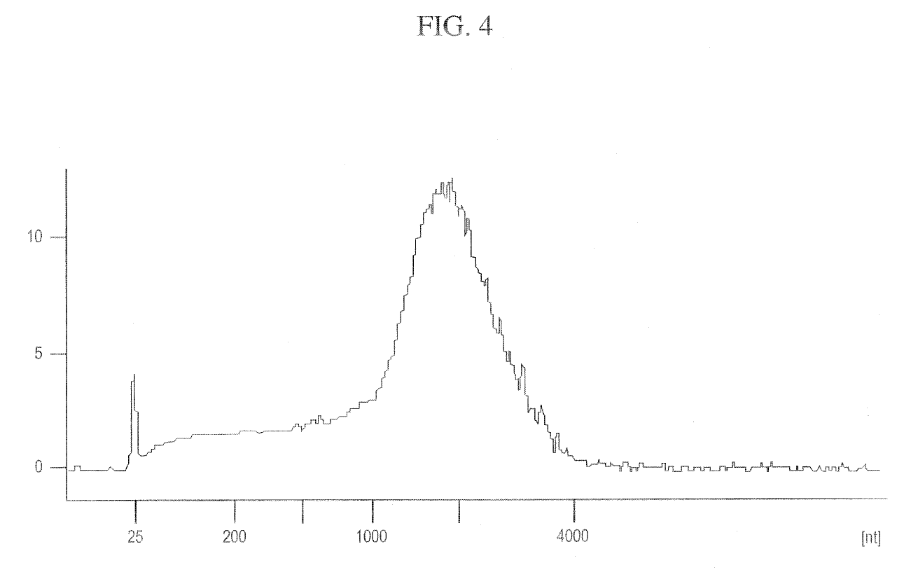

In another method embodiment, 10 mL of anti-coagulated blood was passed through a LDF to capture the WBCs, the LDF with captured WBCs was then treated with RNAlater® Solution, and subsequently flushed with Lysis Solution to release the RNA as described in the previous example. The LDF was then combined with 2 ml of DNase digestion solution (100 μL of DNase 1 (2 units/μL), 200 μL 10× DNase buffer, and 1.7 mL nuclease-free water) to fragment the DNA, as described in previous example. The DNase digestion solution comprising the DNA fragments was collected and passed back over the LDF a second time to maximize the amount of DNA recovered, and the filtrate recovered in 2 aliquots of approximately 0.9 mL each, in 2 mL microfuge tubes. To inactivate the DNase, 10 μL of 0.5 M EDTA pH 8 was added to each tube and mixed by vortexing, and the tubes were then incubated in a 75° C. heatblock for 10 min. To isolate the DNA fragments, the tubes were centrifuged briefly to collect condensate, and then 180 μL of 3 M sodium acetate pH 5.5 (Ambion cat #9740) was added to each tube and mixed by vortexing, followed by addition of 1 mL of isopropanol to each tube and vortex mixing. The tubes were placed in a dry ice/ethanol bath for 10 min to precipitate the DNA fragments, and the DNA was recovered by centrifuging the tubes for 15 min at 13,200 rpm. The supernatant fluid was removed and the pellets of DNA washed by adding 1.8 mL of 70% ethanol to each, vortexing briefly to dislodge the pellets from the bottom of the tube, and re-centrifuging the tubes for 2 minutes longer at 13,200 rpm. The supernatant fluid was thoroughly removed and the pellets resuspended in 100 μL of 0.1 mM EDTA in nuclease-free water. To completely solubilize the precipitated DNA, the tubes were incubated for 2 minutes at 80° C. with intermittent vortexing, and then the contents of the 2 tubes were pooled into a single tube. One μL of the isolated DNA was analyzed on an Agilent 2100 Bioanalyzer, as before. The results, shown in FIG. 4, indicate that the majority of the DNA fragments range in size from 1000 to over 3000 nucleotides in length. The obtained DNA was also analyzed on a Nanodrop spectrophotometer and the resulting concentration was 301 ng/μL, corresponding to a final yield of 60.2 μg. The 260/280 ratio was 1.83.

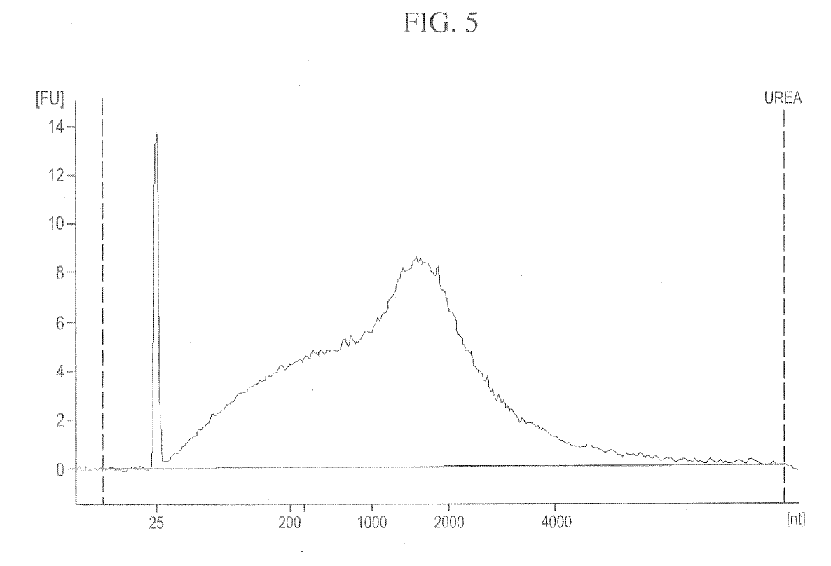

In yet another embodiment, 10 mL of anti-coagulated blood was combined with an LDF and at least some of the nucleated cells were retained on the filter, as described above. The capture surface comprising the retained cells was treated with RNAlater® Solution to stabilize the RNA in the retained cells, as described above. Two mL of a lysis solution 8 M urea and 250 mM NaCl was combined with the capture surface to lyse the retained cells and release the RNA, essentially as described above. The DNA was obtained from the capture surface using 2 mL of a nuclease digestion solution comprising 50 U/mL of DNAse 1, delivered through a 3 mL syringe over period of about 10 seconds, essentially as described above. The nuclease digestion comprising the obtained DNA fragments was collected in a 15 mL plastic tube containing 1 gram of solid GuSCN, and after shaking the tube to dissolve the GuSCN, the prep was mixed with half-volume of 100% ethanol. The DNA was recovered by solid-phase extraction onto a silica filter (from RNAqueous® kit; Ambion) as described in the first example. The DNA was eluted in 200 μL of 0.1 mM EDTA in nuclease-free water, heated to 80° C. and collected. One μL of the isolated DNA was analyzed on an Agilent 2100 Bioanalyzer, as before. As shown in FIG. 5, a large proportion of the obtained DNA fragments (47%) were between one and four kilobases in length. The isolated DNA was also analyzed using a Nanodrop spectrophotometer and the concentration was determined to be 112.71 ng/μL and the A260/280 ratio was 1.9.

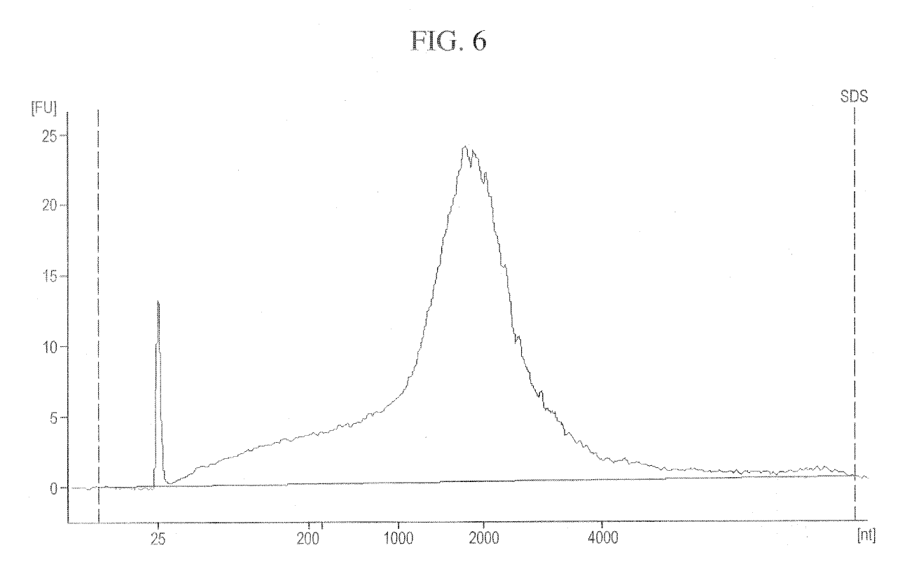

In another embodiment of the current teachings, 10 mL of anti-coagulated blood was combined with an LDF capture surface and the leukocytes were retained, essentially as described above. The capture surface comprising the retained was then combined with RNAlater® Solution to stabilize the RNA, and the capture surface was combined with 2 ml of a lysis solution comprising 2% sodium dodecyl sulfate (SDS), lysing the retained cells and releasing the RNA. The DNA was subsequently obtained from the capture surface by combining the capture surface with 2 mL of a nuclease digestion solution comprising 50 U/mL of DNAse 1, delivered through a 3 mL syringe over period of approximately 10 seconds to fragment the DNA. The nuclease digestion solution comprising the DNA fragments was collected in a 15 mL plastic tube containing 1 gram of solid GuSCN, and after shaking the tube to dissolve the GuSCN, the prep was mixed with half-volume of 100% ethanol. To isolate the DNA fragments, this solution was combined with and purified on a silica filter from an Ambion RNAqueous® Kit, essentially as described above. The DNA was eluted in 200 μL of 0.1 mM EDTA in nuclease-free water, heated to 80° C. and 1 μL of the isolated DNA was analyzed using an Agilent 2100 Bioanalyzer. As shown in FIG. 6, the majority of the isolated DNA fragments (72%) were in the 1 kb to 4 kb size range. The isolated DNA was also analyzed using a Nanodrop spectrophotometer and the yield was determined to be 162.46 ng/μL and the A260/280 ratio was 1.92.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

Although the disclosed teachings have been described with reference to various applications, methods, and compositions, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

What is claimed is:

1. A method for obtaining deoxyribonucleic acid (DNA) from cells comprising:
combining the cells comprising ribonucleic acid (RNA) and DNA with a capture surface under conditions suitable to retain at least some of the cells on the capture surface,
lysing the retained cells to release at least some of the RNA from the lysed retained cells, and
combining the capture surface with a nuclease to obtain at least some of the DNA from the capture surface.

2. The method of claim 1, further comprising inactivating the nuclease after obtaining the DNA.

3. The method of claim 2, wherein the inactivating the nuclease comprises an inactivating agent.

4. The method of claim 3, wherein the inactivating comprises combining the obtained DNA with a solid chaotropic salt.

5. The method of claim 2, wherein the inactivating comprises heating.

6. The method of claim 1, wherein the released RNA is isolated, the obtained DNA is isolated, or the released RNA and the obtained DNA are isolated.

7. The method of claim 6, wherein the isolating comprises a solid phase, an organic extraction step, or both.

8. The method of claim 1, wherein the cells are nucleated cells in whole blood or a blood fraction and further comprising isolating the released RNA, the obtained DNA or the released RNA and the obtained DNA.

9. The method of claim 1, wherein the nuclease comprises DNase I, benzonase, S1 nuclease, mung bean nuclease, micrococcal nuclease, a restriction endonuclease, or combinations thereof.

10. The method of claim 1, wherein the capture surface comprises silica, at least one organic polymer, or combinations thereof.

11. A method for sequentially obtaining RNA and DNA from leukocytes comprising,
combining a solution comprising the leukocytes with a capture surface under conditions suitable to retain at least some of the leukocytes on the capture surface,
lysing the retained leukocytes to release at least some of the RNA from the lysed retained leukocytes,
combining the capture surface with a nuclease to obtain at least some of the DNA from the capture surface, and
inactivating the nuclease.

12. The method of claim 11, wherein the capture surface comprises a leukocyte depletion filter.

13. The method of claim 11, wherein the solution comprising the leukocytes comprises whole blood or a blood fraction.

14. The method of 11, wherein the nuclease comprises wherein the nuclease comprises DNase I, benzonase, S1 nuclease, mung bean nuclease, micrococcal nuclease, a restriction endonuclease, or combinations thereof.

15. The method of claim 11, wherein the inactivating the nuclease comprises an inactivating agent.

16. The method of claim 15, wherein the inactivating comprises combining the obtained DNA with a solid chaotropic salt.

17. The method of claim 11, wherein the inactivating comprises heating.

18. The method of claim 11, wherein the released RNA is isolated, the obtained DNA is isolated, or the released RNA and the obtained DNA are isolated.

19. The method of claim 18, further comprising using the isolated RNA for gene expression profiling, miRNA expression profiling, or both gene expression profiling and miRNA expression profiling.

20. The method of claim 18, further comprising using the isolated DNA in the polymerase chain reaction (PCR), methylation analysis, genotyping, or combinations thereof.
